(19) United States
(12) Patent Application Publication
(10) Pub. No.: US 2024/0285655 A1
(43) Pub. Date: Aug. 29, 2024

(54) METHODS FOR TREATING INFLAMMATORY AND/OR AUTOIMMUNE SYMPTOMS ASSOCIATED WITH BIOLOGIC AND/OR MRNA VACCINE ADMINISTRATION

(71) Applicant: PKZ, LLC, Boca Raton, FL (US)

(72) Inventors: J. Phillip Kennedy, Grand Rapids, MI (US); Karyemaître Aliffe, Olympia, WA (US)

(21) Appl. No.: 18/586,450

(22) Filed: Feb. 24, 2024

Related U.S. Application Data
(60) Provisional application No. 63/448,022, filed on Feb. 24, 2023.

(51) Int. Cl. A61K 31/00; A61K 31/015; A61K 31/05; A61K 31/12; A61K 31/138; A61K 31/167; A61K 31/20; A61K 31/352; A61K 31/375; A61K 31/4706; A61K 31/593; A61K 31/704; A61K 33/06; A61K 35/10; A61K 36/185; A61K 38/40; A61K 38/48

(52) U.S. Cl. CPC A61K 31/658; A61K 31/015; A61K 31/05; A61K 31/12; A61K 31/138; A61K 31/167; A61K 31/20; A61K 31/352; A61K 31/375; A61K 31/4706; A61K 31/593; A61K 31/704; A61K 33/06; A61K 35/10; A61K 36/185; A61K 38/40; A61K 38/482

(57) ABSTRACT

Methods for treating inflammatory, autoinflammatory or autoimmune symptoms associated with administration of a biologic (e.g., a vaccine) are described herein. Certain methods include treating side effects of mRNA based vaccines such as COVID-19 vaccines.

### CROSS-REFERENCE TO RELATED APPLICATIONS

This application claims priority from U.S. Provisional Patent Application No. U.S. 63/448,002 filed 24 Feb. 2023, the entire contents of which are incorporated herein by reference.

### FIELD OF THE INVENTION

The present invention relates to the treatment of inflammatory, autoinflammatory and/or autoimmune symptoms associated with the administration of biologics, such as vaccines. In particular, the present invention relates to the use of compositions comprising an immunosuppressant, immunomodulator, anti-autoimmune, anti-allergy and/or anti-inflammatory agent in conjunction with a biologic to treat or prevent any associated inflammatory, autoinflammatory and/or autoimmune symptoms.

### BACKGROUND

Human gene therapy seeks to modify or manipulate the expression of a gene or to alter the biological properties of living cells for therapeutic use. Gene therapy is a technique that modifies a person's genes to treat or cure disease. Gene therapies can work by several mechanisms. Gene therapy is a medical intervention based on modification of the genetic material of living cells. Cells may be modified ex vivo for subsequent administration or may be altered in vivo by gene therapy products given directly to the subject.

mRNA-based therapeutics and vaccines are a relatively new category of gene therapy products, or “biopharmaceuticals”, that have recently been included on the recommended immunization lists of public health organizations worldwide. mRNA-based therapeutics are a breakthrough class of biologic medicines applicable to a growing number of diseases and conditions as preventative, palliative, and curative therapies. However, with the rapid approval of mRNA vaccines to treat the COVID-19 pandemic, there is much that is yet to be understood about how these vaccines interact with the immune system and their long term effects.

With billions of doses of these mRNA vaccines administered worldwide over the past 2-3 years, there is an unmet need for methods to treat or prevent immune-related side effects associated with their administration.

### SUMMARY OF THE INVENTION

In one aspect, the present invention provides a method of treating or preventing an inflammatory, autoinflammatory and/or autoimmune symptom associated with administration of biologic to a subject, the method comprising administering to the subject an effective amount of a composition containing an immunosuppressant, immunomodulator, anti-autoimmune, anti-allergy, anti-inflammatory agent, or a combination thereof.

In another aspect, the present invention provides a method of treating or preventing an inflammatory, autoinflammatory and/or autoimmune symptom associated with administration of a COVID-19 vaccine to a subject, the method comprising administering to the subject an effective amount of a composition containing an immunosuppressant, immunomodulator, anti-autoimmune, anti-allergy, anti-inflammatory agent, or combinations thereof.

In another aspect, the present invention provides use of an immunosuppressant, immunomodulator, anti-autoimmune, anti-allergy, anti-inflammatory agent, or a combination thereof, in the manufacture of a medicament for treating or preventing an inflammatory, autoinflammatory and/or autoimmune symptom associated with administration of a biologic to a subject.

In another aspect, the present invention provides use of an immunosuppressant, immunomodulator, anti-autoimmune, anti-allergy, anti-inflammatory agent, or a combination thereof, in the manufacture of a medicament for treating or preventing an inflammatory, autoinflammatory and/or autoimmune symptom associated with administration of a COVID-19 vaccine to a subject.

### DETAILED DESCRIPTION

Various aspects now provided will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of values that fall within 10%, 9%, 8%, 7%, 6%, 5%, 4%, 3%, 2%, 1%, or less in either direction (greater than or less than) of the stated value unless otherwise stated or otherwise evident from the context (except where such number would exceed 100% of a possible value). In a particular embodiment, the term “about” means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein mean introducing the pharmaceutical composition into the system of the subject in need of treatment, and refer to directly administering a compound (also referred to as an agent of interest) or pharmaceutically acceptable salt of the compound (agent of interest) or a composition to a subject. When the pharmaceutical composition is provided in combination with one or more other active agents, “administration” and its variants are each understood to include concurrent and/or sequential introduction of the pharmaceutical composition and the other active agents.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound or composition that, when administered to a subject, is sufficient to treat or reduce an inflammatory, autoinflammatory and/or autoimmune symptom associated with administration of a biologic (e.g., a vaccine) to a subject. The therapeutically effective amount to be administered will be governed by such considerations, and may be either an incremental maximum tolerated dose or the minimum amount necessary to ameliorate, cure, prevent or treat the symptom. The actual amount which comprises the “effective amount” or “therapeutically effective amount” may also vary depending on a number of symptoms including, but not limited to, the severity of the symptoms, the size and health of the patient, and the route of administration. A skilled medical practitioner can readily determine the appropriate amount using methods known in the medical arts.

The phrase “pharmaceutically acceptable” is employed herein to refer to those agents of interest/compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopcia for use in mammals (e.g., animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts may be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylate, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethane sulfonic, sulfanilic, cyclohexyl aminosulfonic, alginic, 3-hydroxybutyric, galactaric, and galacturonic acid. The preparation of the pharmaceutically acceptable salts described above and other typical pharmaceutically acceptable salts is more fully described by Berge et al., “Pharmaceutical Salts,” J. Pharm. Sci., 1977:66:1-19.

The term “patient” and “subject” are interchangeable and may be taken to mean any living organism which may be treated with compounds of the present invention. As such, the terms “patient” and “subject” may include, but are not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The terms “treat”, “treating” or “treatment” are used herein, for instance, in reference to methods of treating an inflammatory, autoinflammatory and/or autoimmune symptom associated with administration of a biologic (e.g., a vaccine), and generally include the administration of a compound or composition which reduces the frequency of, or delays the onset of, the inflammatory, autoinflammatory and/or autoimmune symptoms in a subject relative to a subject not receiving the compound or composition. This may include reversing, reducing, or arresting the symptoms, clinical signs, and underlying pathology of a condition in a manner to improve or stabilize a subject's condition. The terms “treat” “treating” or “treatment” are used herein may also encompass the prevention of an inflammatory, autoinflammatory and/or autoimmune symptom associated with administration of a biologic (including a vaccine). The term “prevention” (and grammatical variations thereof) as used herein refer to administering a medicament in order to avert or forestall the appearance of one or more inflammatory, autoinflammatory and/or autoimmune symptoms associated with administration of a biologic. The person of ordinary skill in the medical art recognizes that the term “prevent” is not an absolute term. In the medical art, it is understood to refer to the prophylactic administration of a drug to substantially diminish the likelihood or seriousness of a condition, or symptom of the condition and this is the sense intended in this disclosure. As used in a standard text in the field, the Physician's Desk Reference, the terms “prevent”, “preventing,” and “prevention” with regard to a condition refer to averting the cause, effects, symptoms or progression of a condition prior to the condition fully manifesting itself.

The terms “biologic” or “biologics” are used herein in reference to any pharmaceutical drug product manufactured in, extracted from, synthesized from, or semi-synthesized from biological sources. “Biologics” encompasses vaccines, whole blood, blood components, allergenics, somatic cells, gene therapies, tissues, recombinant therapeutic protein, and living medicines used in cell therapy. “Biologics” may be composed of sugars, proteins, nucleic acids, or complex combinations of these substances, or may be living cells or tissues. They (or their precursors or components) may be isolated from living sources-human, animal, plant, fungal, or microbial and may be used in both human and animal medicine.

As used herein, the term “alkyl” or “alkyl group” means a linear or branched, saturated hydrocarbon including one or more carbon atoms (e.g., one, two, three, four, five, six, seven, eight, nine, ten, or more carbon atoms), which is optionally substituted. The notation “C₁-C₁₀ alkyl” means an optionally substituted linear or branched, saturated hydrocarbon including 1-10 carbon atoms, “C₁-C₆ alkyl” means an optionally substituted linear or branched, saturated hydrocarbon including 1-6 carbon atoms, “C₁-C₃ alkyl” means an optionally substituted linear or branched, saturated hydrocarbon including 1-3 carbon atoms, and the like. Unless otherwise specified, an alkyl group described herein refers to both unsubstituted and substituted alkyl groups. Examples of suitable alkyl groups may include, but are not limited to, methyl, ethyl, 1-propyl, isopropyl, 1-butyl, 2-butyl, isobutyl, sec-butyl, tert-butyl, pentyl, hexyl, heptyl, octyl, nonyl, decyl, and the like.

As used herein, the term “alkenyl” or “alkenyl group” means a linear or branched hydrocarbon including two or more carbon atoms (e.g., two, three, four, five, six, seven, eight, nine, = or more carbon atoms) and at least one double bond, which is optionally substituted. Unless indicated otherwise, the stercochemistry about each double bond may be independently cis or trans, or E or Z, as appropriate. The notation “C₂-C₁₀ alkenyl” means an optionally substituted linear or branched hydrocarbon including 2-10 carbon atoms and at least one carbon-carbon double bond, “C₂-C₆ alkenyl” means an optionally substituted linear or branched hydrocarbon including 2-6 carbon atoms and at least one carbon-carbon double bond, “C₂-C₃ alkenyl” means an optionally substituted linear or branched hydrocarbon including 2-3 carbon atoms and at least one carbon-carbon double bond, and the like. An alkenyl group may include one, two, three, four, or more carbon-carbon double bonds. For example, C₁₀ alkenyl may include one or more double bonds. Unless otherwise specified, an alkenyl group described herein refers to both unsubstituted and substituted alkenyl groups.

As used herein, the term “alkynyl” or “alkynyl group” means a linear or branched hydrocarbon including two or more carbon atoms (e.g., two, three, four, five, six, seven, eight, nine, ten, or more carbon atoms) and at least one carbon-carbon triple bond, which is optionally substituted. The notation “C₂-C₁₀ alkynyl” means an optionally substituted linear or branched hydrocarbon including 2-10 carbon atoms and at least one carbon-carbon triple bond, “C₂-C₆ alkynyl” means an optionally substituted linear or branched hydrocarbon including 2-6 carbon atoms and at least one carbon-carbon triple bond. An alkynyl group may include one, two, three, four, or more carbon-carbon triple bonds. For example, C₁₀ alkynyl may include one or more carbon-carbon triple bonds. Unless otherwise specified, an alkynyl group described herein refers to both unsubstituted and substituted alkynyl groups.

As used herein, the term “alkoxy” or “alkoxyl group” means a chemical substituent of formula —OR, where R is an alkyl group as defined herein (e.g., O—C₁-C₁₀ alkyl, O—C₁₋₆ alkyl or O—C₁-C₃ alkyl), unless otherwise specified. Exemplary alkoxy groups include methoxy, ethoxy, propoxy (e.g., n-propoxy and isopropoxy), t-butoxy, and the like.

As used herein, the term “aryl” refers to an optionally substituted monocyclic, or fused polycyclic, aromatic carbocycle (i.e., a ring structure having ring atoms that are all carbon). The aryl group may have from 6-10 atoms per ring, denoted C₆₋₁₀ aryl. Examples of suitable aryl groups may include, but are not limited to, phenyl, naphthyl, phenanthryl. As used herein, the term “aryl” is also intended to encompass optionally substituted partially saturated bicyclic aromatic carbocyclic moiety in which a phenyl and a cycloalkyl or cycloalkenyl group are fused together to form a cyclic structure, such as tetrahydronaphthyl, indenyl or indanyl. The aryl group may be a terminal group or a bridging group.

As used herein, the term “aryloxy” or “aryloxyl group” means a chemical substituent of formula —OR, where R is an aryl group as defined herein (e.g., O—C₆-C₁₀ aryl), unless otherwise specified.

As used herein, the term “arylalkyl” or “arylalkyl group” means a chemical substituent of formula —R^{a}—R^{b}, where R^{a} is an alkyl group as defined herein and R^{b} is an aryl group as defined herein (e.g., —C₁-C₁₀ alkyl-C₁-C₁₀ aryl), unless otherwise specified.

As used herein, the term “amine” means a chemical substituent of formula —NR^{c}R^{d}, wherein R^{c} and R^{d} are each independently an alkyl group as defined herein (e.g., N(C₁-C₁₀ alkyl)(C₁-C₁₀ alkyl)), unless otherwise specified. Exemplary amine groups include, dimethylamine, diethylamine, diisopropylamine, and the like.

As used herein, the term “amino acid” refers to any natural or non-natural amino acid, including but not limited to: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, homoserine, and norleucine. As used herein, the term “amino acid ester” refers to any amino acid in which the hydrogen atom of the acidic hydroxyl group of that acid is replaced by an “alkyl” group as defined elsewhere herein.

A reference to a percentage (%) content throughout this specification is to be taken to mean a percentage by weight, denoted “% w/w” or “wt. %”. Unless otherwise specified, any reference to a percentage by weight of a component of a composition or formulation described herein refers to the percentage by weight of the specified component with respect to the total components of the composition or formulation. Certain components of the compositions described herein may be provided as a liquid. In such cases, it may be more convenient to measure the amount of a liquid component as a volume (e.g., in mL). A skilled person will be able to readily determine the weight of a liquid component given its volume by multiplying the volume (in mL) by the density of the liquid (in g/mL), for example, as measured at 25° C.

The terms “composition” and “formulation” are used interchangeably throughout this specification and have the same meaning.

By hereby reserving the right to proviso out or exclude any individual members of any such group, including any sub-ranges or combinations of sub-ranges within the group, that may be claimed according to a range or in any similar manner, less than the full measure of this disclosure may be claimed for any reason. Further, by hereby reserving the right to proviso out or exclude any individual substituents, analogs, compounds, ligands, structures, or groups thereof, or any members of a claimed group, less than the full measure of this disclosure may be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

The reference to any prior art in this specification is not, and should not be taken as, an acknowledgment or any form of suggestion that that prior art forms part of the common general knowledge.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Historically, biologic medicines, or biopharmaceuticals, have brought significant advancements in medical care and human health overall. Two examples of this are antibiotics and insulin replacement therapies. At the time of their introduction into the medical armamentarium, however, both contained critical impurities that provoked severe reactions in numerous patients, often resulting in death. As a novel class of biopharmaceuticals, mRNA therapeutic agents produced at industrial scale inevitably contain trace impurities as well. These can include nucleic acid fragments and synthetic molecular by-products, in addition to the adjuvant (vaccine ingredients designed to trigger a stronger immune response) and excipient materials included in the formulations. Many biopharmaceuticals, including mRNA vaccines, also envelop the mRNA in lipid nanoparticles (LNPs). LNPs function to increase the stability and half-life of mRNA, improve transport to cells, and also act as adjuvants. At present, the ability to detect and quantify these nanostructures within the human body is lacking; therefore, their tissue distribution, persistence and local effects cannot be tracked or managed. Their minuscule size also enables LNPs to pass freely into and distribute through various bodily tissues and organs (e.g., the lining of the intestines and blood vessels, the lungs, the heart, the reproductive organs, etc.). Thus, the manufacturing processes and excipients used to prepare biologic medicines, including mRNA vaccines, have the potential to trigger inflammatory and immune responses, which can precipitate tissue and organ inflammation characteristic of autoimmune conditions.

Thus, the present invention relates generally to methods of treating or preventing an inflammatory, autoinflammatory and/or autoimmune symptom associated with administration of a biologic (e.g., a vaccine) to a subject, the methods comprising administering to the subject an effective amount of an immunosuppressant, immunomodulator, anti-autoimmune, anti-allergy, anti-inflammatory agent, or a combination thereof, or a pharmaceutical composition comprising the same.

The present invention also provides use of an immunosuppressant, immunomodulator, anti-autoimmune, anti-allergy, anti-inflammatory agent, or a combination thereof, in the manufacture of a medicament for treating or preventing an inflammatory, autoinflammatory and/or autoimmune symptom associated with administration of a biologic (e.g., a vaccine) to a subject.

The present invention also provides use of an immunosuppressant, immunomodulator, anti-autoimmune, anti-allergy, anti-inflammatory agent, or a combination thereof, or a pharmaceutical composition comprising the same, for treating or preventing an inflammatory, autoinflammatory and/or autoimmune symptom associated with administration of a biologic (e.g., a vaccine) to a subject.

Various embodiments of the invention are directed to methods for treating inflammatory and/or autoimmune disorders associated with the administration of gene therapy and/or therapies, including biologics, including all agents administered to function as any form of vaccine. In some cases, autoimmune-like symptoms may manifest following administration of mRNA-based vaccines, such as COVID-19 vaccines. Non-limiting examples of COVID-19 vaccines include Spikevax® (Moderna) and Comirnaty® (Pfizer). In other embodiments, the vaccine may be an iDC vaccine or an IFN-α vaccine (e.g., IFNα Kinoid).

The present invention may be particularly suitable for treating or preventing an inflammatory, autoinflammatory and/or autoimmune symptom associated with administration of a COVID-19 vaccine to a subject. Thus, the present invention also provides a method of treating or preventing an inflammatory, autoinflammatory and/or autoimmune symptom associated with administration of a COVID-19 vaccine in a subject, the method comprising administering to the subject an effective amount of an immunosuppressant, immunomodulator, anti-autoimmune, anti-allergy, anti-inflammatory agent, or a combination thereof, or a pharmaceutical composition comprising the same.

The present invention also provides use of an immunosuppressant, immunomodulator, anti-autoimmune, anti-allergy, anti-inflammatory agent, or a combination thereof, in the manufacture of a medicament for treating or preventing an inflammatory, autoinflammatory and/or autoimmune symptom associated with administration of a COVID-19 vaccine to a subject.

The present invention also provides use of an immunosuppressant, immunomodulator, anti-autoimmune, anti-allergy, anti-inflammatory agent, or a combination thereof, or a pharmaceutical composition comprising the same, for treating or preventing an inflammatory, autoinflammatory and/or autoimmune symptom associated with administration of a COVID-19 vaccine to a subject.

Symptoms associated with vaccines may begin to manifest any time following vaccine administration. For example, in some cases, patients administered such vaccines may exhibit inflammatory, autoinflammatory and/or autoimmune-like symptoms immediately following administration. In other embodiments, such symptoms may manifest within 1 month after administration, 1 month to 6 months after administration, 4 months to 12 months after administration, 9 months to 18 months after administration, 1 year to 3 years after administration, 2 years to 5 years after administration, or any range or individual time period encompassed by these example ranges. The methods disclosed herein may include administering an effective amount of a pharmacologic agent, including an immunosuppressant and/or immunomodulator and/or anti-autoimmune and/or anti-allergy and/or anti-inflammatory agent or a composition containing an effective amount of an immunosuppressant and/or immunomodulator and/or anti-autoimmune and/or anti-allergy and/or anti-inflammatory agent before, concurrently, or following administration of a biopharmaceutical or biologic agent, including a vaccine, and in some embodiments an mRNA-based therapeutic (i.e., a drug or medication) or vaccine. In some embodiments, the methods may include administering an effective amount of an immunosuppressant and/or immunomodulator and/or anti-autoimmune and/or anti-allergy and/or anti-inflammatory agent or a composition containing an effective amount of an immunosuppressant and/or immunomodulator and/or anti-autoimmune and/or anti-allergy and/or anti-inflammatory agent before, concurrently or following the onset of symptoms associated with an autoimmune and/or inflammatory disorder.

Inflammatory, autoinflammatory and/or autoimmune symptoms (also referred to herein as “autoimmune-like symptoms”) include any symptoms known to be associated with autoimmune diseases including, but are not limited to, joint pain and stiffness; muscle aches, pains, or weakness, fever; malaise; cutaneous manifestations (e.g., butterfly-shaped rash across the nose and checks; other skin rashes); unusual weight loss or weight gain; anemia; neurological or neuropsychiatric manifestations (e.g., trouble thinking, memory problems, confusion, depression, headache, seizures, strokes); kidney problems (e.g., nephritis, e.g., glomerulonephritis); chest pain; sun or light sensitivity; hair loss; Raynaud's phenomenon; vascular lesions or other vascular manifestations (e.g., Raynaud's phenomenon, nail fold telangiectasia and infarct, splinter hemorrhages, chilblain LE, acquired C1 esterase deficiency, vasculitis, urticarial vasculitis, purpura, thrombophlebitis, livedo reticularis, antiphospholipid syndrome, Degos syndrome and calcinosis); Guillain-Barré syndrome, Functional Neurological Disorder (FND), as well as biological concomitants of lupus as disclosed herein or as known in the art (e.g., immune complexes, elevated levels of cytokines (e.g., interferons (e.g., Type I interferons, e.g., IFN-α and/or IFN-β); interleukins (e.g., IL-6, IL-8, IL-1, and IL-18) and TNF-α), elevated levels of antibodies associated with lupus (e.g., antinuclear antibodies (e.g., anti-Smith antibodies, anti-double stranded DNA (dsDNA) antibodies, anti-U1 RNP, SS-a (or anti-Ro), SS-b (or anti-La)), antiphospholipid antibodies, anti-ssDNA antibodies, anti-histone antibodies, or anticardiolipin antibodies), overexpression of interferon-inducible genes, and the like, and combinations thereof. Symptoms may be assessed using assays and scales (e.g., Systemic Lupus Activity Measure-Revised (SLAM-R) disclosed and the like and combinations thereof.

In some embodiments, the inflammatory, autoinflammatory and/or autoimmune-like symptoms may include nephritis (e.g., glomerulonephritis, interstitial nephritis, perivascular inflammation, or protein cast severity), proteinuria, or spleen inflammation. In certain embodiments, the symptoms may include glomerulonephritis or an immune complex. In some embodiments, the inflammatory- and/or autoimmune-like symptoms may include overexpression of pro-inflammatory cytokines (e.g., IFN-α, TNF-α, IL-6, IL-8, IL-1, IL-17, IL-23).

Suitable immunosuppressant and/or immunomodulator and/or anti-autoimmune and/or anti-allergy and/or anti-inflammatory agents will be apparent to those skilled in the art and are not limited to particular agents. For example, embodiments are not limited to a particular anti-inflammatory agent. Exemplary anti-inflammatory agents include, but are not limited to, retinoid and/or salicylate compounds, anti-fibrotics, corticosteroids, anti-inflammatories, immunosuppressants, chemotherapeutic agents, anti-metabolites, and immunomodulators.

For example, in some embodiments, the anti-inflammatory agent may be an anti-interferon agent such as AGS-009, Rontalizumab (rhuMAb IFNalpha), Vitamin D3, Sifalimumab (MEDI-545), AMG 811, IFNα Kinoid, or CEP33457. In some embodiments, the anti-inflammatory agent may be an anti-B-cell agent such as Epratuzumab, LY2127399, Ocrelizumab, Atacicept, A-623, or SBI-087 or an anti-T-cell agent such as AMG557. In some embodiments, the anti-inflammatory agent may be an immunomodulatory or immunosuppressant agent such as laquinimod, rapamycin, cyclophosphamide (Cytoxan), azathioprine (Imuran, Azasan), mycophenolate (Cellcept), leflunomide (Arava) or methotrexate (Trexall), an anti-interleukin therapy such as CNTO 136. In further embodiments, the anti-inflammatory agent may be tamibarotene, N-acetylcysteine, CDP7657, hydroxychloroquine, B cell depletion, or anti-CD20 therapy (e.g., rituximab), a proteasome inhibitor (e.g., carfilzomib or bortezomib) or an immunoproteasome inhibitor (e.g., ONX 0914), a toll-like receptor inhibitor (e.g., hydroxychloroquine, IMO-3100, or DV1179), a nonsteroidal anti-inflammatory drug (NSAID), a corticosteroid, and the like and combinations thereof.

In further embodiments, the anti-inflammatory agent may be, for example, an immunosuppressant (e.g., methotrexate, azathioprinc (Imuran®), cyclosporine, mycophenolate mofetil (Cellcept®), and cyclophosphamide (Cytoxan®)), T-cell-directed therapy (e.g., halofuginone, basiliximab, alemtuzumab, abatacept, rapamycin), B-cell directed therapy (e.g., rituximab), autologous hematopoietic stem cell transplantation, a chemokine ligand receptor antagonist (e.g., an agent that targets the CXCL12/CSCR4 axis such as AMD3100), a DNA methylation inhibitor (e.g., 5-azacytidine), a histone dactylase inhibitor (e.g., trichostatin A), a statin (e.g., atorvastatin, simvastatin, pravastatin), an endothelin receptor antagonist (e.g., Bosentan®), a phosphodiesterase type V inhibitor (e.g., Sildenafil®), a prostacyclin analog (e.g., trepostinil), an inhibitor of cytokine synthesis and/or signaling (e.g., Imatinib mesylate, Rosiglitazone, rapamycin, anti-transforming growth factor ß1 (anti-TGFß1) antibody, mycophenolate mofetil, an anti-IL-6 antibody (e.g., tocilizumab)), corticosteroids, nonsteroidal anti-inflammatory drugs, light therapy, blood pressure medications (e.g., ACE inhibitors), Jak2 inhibitor (e.g., INCB018424, XL019, TG101348, or TG101209), an immunomodulator, (e.g., an thalidomide, lenalidomide, or panolinomide), hydroxyurca, an androgen, erythropoictic stimulating agents, prednisone, danazol, HDAC inhibitors, eplerenone, furosemide, pycnogenol, spironolactone, TcNC100692, torasemide (e.g., prolonged release form of torasemide), cyclosporine, cyclosporine A, daclizumab, everolimus, gadofoveset trisodium (ABLAVAR®), imatinib mesylate (GLEEVEC®), matinib mesylate, methotrexate, mycophenolate mofetil, prednisone, sirolimus, spironolactone, STX-100, tamoxifen, TheraCLEC™, Bosentan (Tracleer), p144, pentoxifylline; pirfenidone; pravastatin, STI571, Vitamin E, ALTU-135, bucelipase alfa (INN), DCI1020, EUR-1008 (ZENPEP™), ibuprofen, Lym-X-Sorb powder, pancrease MT, pancrelipase (e.g., pancrelipase delayed release), pentade canoic acid (PA), repaglinide, TheraCLEC™, triheptadecanoin (THA), ULTRASE MT20, ursodiol, 18-FDG, AB0024, ACT-064992 (macitentan), aerosol interferon-gamma, aerosolized human plasma-derived alpha-1 antitrypsin, alphal-proteinase inhibitor, ambrisentan, amikacin, amiloride, amitriptyline, anti-Pseudomonas IgY gargle, ARIKACE™, AUREXIS® (tefibazumab), AZAPRED, azathioprine, azithromycin, azithromycin, AZLI, aztreonam lysine, BIBF1120, Bio-25 probiotic, bosentan, Bramitob®, calfactant aerosol, captopril, CC-930, ceftazidime, ceftazidime, cholecalciferol (Vitamin D3), ciprofloxacin (CIPRO®, BAYQ3939), CNTO 888, colistin CF, combined Plasma Exchange (PEX), rituximab, and corticosteroids, cyclophosphamide, dapsone, dasatinibi, denufosol tetrasodium (INS37217), dornase alfa (PULMOZYMER), EPI-bNE4, erythromycin, etanercept, FG-3019, fluticasone, FTI, GC1008, GS-9411, hypertonic saline, ibuprofen, iloprost inhalation, imatinib mesylate (GLEEVEC®), inhaled sodium bicarbonate, inhaled sodium pyruvate, interferon gamma-1b, interferon-alpha lozenges, isotonic saline, IWO01, KB001, losartan, lucinactant, mannitol, meropenem, meropenem infusion, miglustat, minocycline, Moli1901, MP-376 (levofloxacin solution for inhalation), mucoid exopolysaccharide P. aeruginosa immune globulin IV, mycophenolate mofetil, n-acetylcysteine, N-acetylcysteine (NAC), NaCl 6%, nitric oxide for inhalation, obramycin, octreotide, oligoG CF-5/20, omalizumab, pioglitazone, piperacillin-tazobactam, pirfenidone, pomalidomide (CC-4047), prednisone, prevastatin, PRM-151, QAX576, rhDNAse, SB656933, SB-656933-AAA, sildenafil, tamoxifen, technetium [Tc-99m] sulfur colloid and Indium [In-111] DTPA, tetrathiomolybdate, thalidomide, ticarcillin-clavulanate, tiotropium bromide, tiotropium RESPIMAT® inhaler, tobramycin (GERNEBCIN®), treprostinil, uridine, valganciclovir (VALCYTE®), vardenafil, vitamin D3, xylitol, zileuton, adefovir dipivoxil, candesartan, colchicine, combined ATG, mycophenolate mofetil, tacrolimus, combined cyclosporine microemulsion and tacrolimus, elastometry, everolimus, FG-3019, Fuzheng Huayu, GI262570, glycyrrhizin (monoammonium glycyrrhizinate, glycine, L-cysteine monohydrochloride), interferon gamma-1b, irbesartan, losartan, oltipraz, ORAL IMPACT®, peginterferon alfa-2a, combined peginterferon alfa-2a and ribavirin, peginterferon alfa-2b (SCH 54031), combined peginterferon alpha-2b and ribavirin, praziquantel, prazosin, raltegravir, ribavirin (REBETOL®, SCH 18908), ritonavir-boosted protease inhibitor, pentoxyphilline, tacrolimus, tauroursodeoxycholic acid, tocopherol, ursodiol, warfarin, 552-02, 5-methyltetrahydrofolate and vitamin B12, Ad5-CB-CFTR, Adeno-associated virus-CFTR vector, albuterol, alendronate, alpha tocopherol plus ascorbic acid, amiloride HCl, aquADEK™, ataluren (PTC124), AZD1236, AZD9668, azithromycin, bevacizumab, biaxin (clarithromycin), BIIL 283 BS (amelubent), buprofen, calcium carbonate, ceftazidime, cholecalciferol, choline supplementation, CPX, cystic fibrosis transmembrane conductance regulator, DHA-rich supplement, digitoxin, cocosahexaenoic acid (DHA), doxycycline, ECGC, ecombinant human IGF-1, educed glutathione sodium salt, ergocalciferol (vitamin D2), fluorometholone, gadobutrol (GADOVIST®, BAY86-4875), gentamicin, ghrelin, glargine, glutamine, growth hormone, GS-9411, H5.001CBCFTR, human recombinant growth hormone, hydroxychloroquine, hyperbaric oxygen, hypertonic saline, IH636 grape seed proanthocyanidin extract, insulin, interferon gamma-1b, loGen (molecular iodine), iosartan potassium, isotonic saline, itraconazole, IV gallium nitrate (GANITE®) infusion, ketorolac acetate, lansoprazole, L-arginine, linczolid, lubiprostone, meropenem, miglustat, MP-376 (levofloxacin solution for inhalation), normal saline IV, Nutropin AQ, omega-3 triglycerides, pGM169/GL67A, pGT-1 gene lipid complex, pioglitazone, PTC124, QAU145, salmeterol, SB656933, SB656933, simvastatin, sitagliptin, sodium 4-phenylbutyrate, standardized turmeric root extract, tgAAVCF, TNF blocker, TOBI, tobramycin, tocotrienol, unconjugated Isoflavones 100, vitamin: choline bitartrate (2-hydroxyethyl)trimethylammonium salt 1:1, VX-770, VX-809, Zinc acetate, anti-interferon therapy, e.g., AGS-009, Rontalizumab (rhuMAb IFNalpha), Vitamin D3, Sifalimumab (MEDI-545), AMG 811, IFNα Kinoid, or CEP33457. In some embodiments, the other therapy is an IFN-α therapy, e.g., AGS-009, Rontalizumab, Vitamin D3, Sifalimumab (MEDI-545) or IFNα Kinoid, prednisone (e.g., oral prednisone)), methotrexate (Trexall, Methotrexate, Rheumatrex), azathioprine (Azasan, Imuran), intravenous immunoglobulin, tacrolimus (Prograf), pimecrolimus, cyclophosphamide (Cytoxan), cyclosporine (Gengraf, Neoral, Sandimmune), hydroxychloroquine (Plaquenil), chloroquine (Aralen), total body irradiation, rituximab (Rituxan), TNF inhibitors (e.g., etanercept (Enbrel), infliximab (Remicade)), AGS-009, Rontalizumab (rhuMAb IFNalpha), Vitamin D3, Sifalimumab (MEDI-545), AMG 811, IFNα Kinoid, or CEP33457, and the like and combinations thereof.

In some embodiments, the retinoid or salicylate anti-inflammatory agent may be, for example, a cannabinoid. “Cannabinoid” refers to a compound that is capable of acting on (e.g., interacting with or binding to) cannabinoid receptors (including CB1 and CB2 receptors, or any endocannabinoid receptor, including endocannabinoid receptor(s) yet to be discovered). A skilled person can readily determine whether a compound interacts with cannabinoid receptor using routine screening assays, such as the ligand binding assays described by Devane et al. (1988) Mol Pharmacol 34:605-613 or by in silico prediction of CB1 affinity (see, e.g., Paulke et al. (2016) Toxicoly Letters 245:1-6). In some embodiments, the cannabinoid is an isolated cannabinoid. As used herein the term “isolated” means a single (individual) target cannabinoid, e.g., a target cannabinoid that is isolated or substantially free of other (non-target) cannabinoids and/or active agents, including plant-isolated cannabinoids. The isolated cannabinoid may have a purity of greater than about 80%, 90%, 95% or 98% by weight. In a particular embodiment, the isolated cannabinoid is a food grade cannabinoid (>95% purity) or a pharmaceutical grade (>98% purity).

Cannabinoids may be produced naturally in the body (endocannabinoids), found in certain plants (phytocannabinoids), other biological organisms, and/or manufactured artificially (synthetic cannabinoids). Cannabinoids produced by cannabis or hemp plants during cultivation and growth (phytocannabinoids) include tetrahydrocannabinolic acid (THCA), cannabinolic acid (CBNA), cannabidiolic acid (CBDA), cannabichromenic acid (CBCA), and cannabigerolic acid (CBGA). In some embodiments, such cannabinoids may be modified as decarboxylated moieties to produce tetrahydrocannabinol (THC), cannabinol (CBN), cannabidiol (CBD), cannabichromene (CBC) and cannabigerol (CBG). Cannabinoids also encompass the metabolites of drugs and other bioactive molecules that bind to, modify the function of, or otherwise interact with the endocannabinoid system, including, but not limited to the metabolite(s) of acetaminophen and/or paracetamol of which N-arachidonoyl phenolamine, a compound also known as “AM404”, is an example. Cannabinoids also encompass the metabolites of these and other cannabinoids that result upon application, administration, or consumption of these compounds by humans to any bodily tissues and/or via any routes of administration, such as, but not limited to, 11-OH-THC and THC-COOH (also known as 11-nor-9-carboxyTHC).

Cannabinoids may be synthesized by chemical or biological methods. In some embodiments, the anti-inflammatory agent may be, for example, a synthetic cannabinoid or cannabidiol such as, for example, Δ-5-cannabidiol (2-(6-isopropenyl-3-methyl-5-cyclohexen-1-yl)-5-pentyl-1,3-benzenediol); Δ-4-cannabidiol (2-(6-isopropenyl-3-methyl-4-cyclohexen-1-yl)-5-pentyl-1,3-benzenediol); Δ-3-cannabidiol (2-(6-isopropenyl-3-methyl-3-cyclohexen-1-yl)-5-pentyl-1,3-benzenediol); Δ-3′7-cannabidiol (2-(6-isopropenyl-3-methylenecyclohex-1-yl)-5-pentyl-1,3-benzenediol); Δ-2-cannabidiol (2-(6-isopropenyl-3-methyl-2-cyclohexen-1-yl)-5-pentyl-1,3-benzenediol); Δ-1-cannabidiol (2-(6-isopropenyl-3-methyl-1-cyclohexen-1-yl)-5-pentyl-1,3-benzenediol); Δ-6-cannabidiol (2-(6-isopropenyl-3-methyl-6-cyclohexen-1-yl)-5-pentyl-1,3-benzenediol), Δ-9-tetrahydrocannabinol (Δ-9-THC), HU-210 (6aR,10aR)-9-(hydroxymethyl)-6,6-dimethyl-3-(2-methyloctan-2-yl)-6H,6aH,7H, 10H,10aH-benzo[c]ochromen-1-ol), cannabidivarin (CBDV), cannabichromene (CBC), cannabichromevarin (CBCV), cannabigerol (CBG), cannabigerovarin (CBGV), cannabielsoin (CBE), cannabicyclol (CBL), cannabivarin (CBV), and cannabitriol (CBT), tetrahydrocannibivarin (THCV), cannabigerol monomethyl ether (CBGM), cannabichromenic acid (CBCA), A-1-tetrahydrocannabinolic acid (THCA), and cannabidiolic acid (CBDA), rimonabant, JWH-018 (naphthalen-1-yl-(1-pentylindol-3-yl)methanone), JWH-073 (naphthalen-1-yl-(1-butylindol-3-yl)methanone), CP-55940 (2-[(1R,2R,5R)-5-hydroxy-2-(3-hydroxypropyl)cyclohexyl]-5-(2-methyloctan-2-yl)phenol), dimethylheptylpyran, HU-331 (3-hydroxy-241R)-6-isopropenyl-3-methyl-cyclohex-2-en-1-yl]-5-pentyl-1,4-benzoquinone), SR144528 (5-(4-chloro-3-methylphenyl)-1-[(4-methylphenyl)methyl]-N-RIS,2S,4R)-1,3,3-trimethylbicyclo[2.2.1]heptan-2-yl]-1H-pyrazole-3-carboxamide), WIN 55,212-2 ((11R)-2-methyl-11-[(morpholin-4-yl)methyl]-3-(naphthalene-1-carbonyl)-9-oxa-1-azatricyclo[6.3.1.04,12]dodeca-2,4(12),5,7-tetraene), JWH-133 ((6aR,10aR)-3-(1,1-dimethylbutyl)-6a,7,10,10a-tetrahydro-6,6,9-trimethyl-6H-dibenzo[b,d]pyran), levonantradol, and AM-2201 (1-[(5-fluoropentyl)-1H-indol-3-yl]-(naphthalen-1-yl)methanone), Δ-8-tetrahydrocannabinol (A-8-THC), 11-hydroxy-A-9-tetrahydrocannabinol, Δ-11-tetrahydrocannabinol, 11-hydroxy-tetrahydrocannabinol, and the like and combinations thereof.

In certain embodiments, the anti-inflammatory agent may be a compound of Formula I:

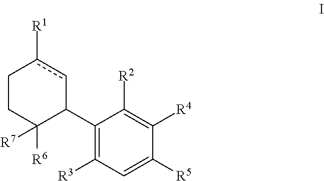

where R¹ is selected from hydrogen, hydroxyl, halogen (F, Cl, Br, I), methyl, linear or branched C₂-C₁₀ alkyl, linear or branched C₂-C₁₀ alkenyl, linear or branched C₂-C₁₀ substituted alkyl, and linear or branched C₂-C₁₀ substituted alkenyl, R² and R³ are each independently selected from hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, R⁴ is selected from hydrogen, hydroxyl, halogen, substituted or unsubstituted alkyl, carboxyl, alkoxy, aryl, aryloxy, arylalkyl, R⁵ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, and R⁶ and R⁷ are each independently selected from hydrogen, hydroxyl, halogen, substituted or unsubstituted alkyl, substituted or unsubstituted alkenyl, substituted or unsubstituted alkynyl, carboxyl, alkoxy, aryl, aryloxy, arylalkyl, amino. The cycloalkyl moiety may or may not include double bonds such as the optional double bond indicated by the dashed line. The compounds of Formula I encompass any stereoisomers, salts, and solvates thereof.

In particular embodiments of Formula I, R¹, R², R³, R⁴, R⁵, R⁶ and R⁷ may each be a functional group, pro-functional group, or isostere thereof, or may be metabolized to an active functional group. In some embodiments, at least R¹, R², R⁴, and R⁵ may be hydrophobic. For example, each of R¹, R², R⁴, and R⁵ may be independently selected from hydrogen, halogen, methyl, linear or branched C₂-C₁₀ alkyl, linear or branched C₂-C₁₀ alkenyl, linear or branched C₂-C₁₀ substituted alkyl, or linear or branched C₂-C₁₀ substituted alkenyl. In some embodiments, R¹ and R³ may be a functional group capable of making a hydrogen bond with a functional group associated with the binding pocket of a RAS protein. For example, R¹ and R³ may be independently selected from hydrogen, hydroxyl, carboxyl, amino, alkyloxy, aryloxy, ketonyl, ester, or ether.

The compounds of various embodiments include anti-inflammatory agents of Formula II:

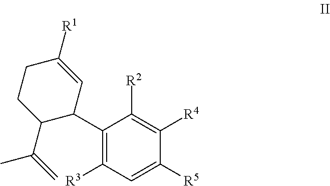

where R¹ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² and R³ are independently selected from hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, R⁴ is hydrogen, hydroxyl, halogen, substituted or unsubstituted alkyl, carboxyl, alkoxy, aryl, aryloxy, arylalkyl, and R⁵ is independently selected from hydrogen, hydroxyl, halogen, methyl, linear or branched C₂-C₁₀ alkyl, linear or branched C₂-C₁₀ alkenyl, linear or branched C₂-C₁₀ substituted alkyl, or linear or branched C₂-C₁₀ substituted alkenyl. The compounds of Formula II encompass any stereoisomers, salts, and solvates thereof.

In particular embodiments of Formula II, R¹, R², R³, R⁴ and R⁵ may each be a functional group, pro-functional group, or isostere thereof, or may be metabolized to an active functional group. In some embodiments, at least R¹, R², R⁴, and R⁵ may be hydrophobic. For example, each of R¹, R², R⁴, and R⁵ be independently selected from hydrogen, halogen, methyl, linear or branched C₂-C₁₀ alkyl, linear or branched C₂-C₁₀ alkenyl, linear or branched C₂-C₁₀ substituted alkyl, or linear or branched C₂-C₁₀ substituted alkenyl. In some embodiments, R¹ and R³ may be a functional group capable of making a hydrogen bond with a functional group associated with the binding pocket of a RAS protein. For example, R¹ and R³ may be independently selected from hydrogen, hydroxyl, carboxyl, amino, alkyloxy, aryloxy, ketonyl, ester, or ether.

In some embodiments, R⁵ may be linear or branched C₂-C₁₀ alkyl. Thus, embodiments include compounds of general formula III:

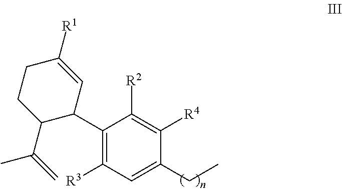

where R¹ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² and R³ are independently selected from hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, R⁴ is selected from hydrogen, substituted or unsubstituted alkyl, carboxyl, alkoxy, aryl, aryloxy, arylalkyl, halo or amino, R² and R³ are independently selected from hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, and n may an integer of 2 to 10 and the like. The compounds of Formula III encompass stereoisomers, salts, and solvates thereof. In some embodiments, R² and R³ may, independently, be a linear or branched, substituted or unsubstituted C₂-C₁₀ acyl having a carboxylic acid terminus thereby producing a dicarboxylic acid, and salts thereof.

In some embodiments, the anti-inflammatory agents may be of general formula IV:

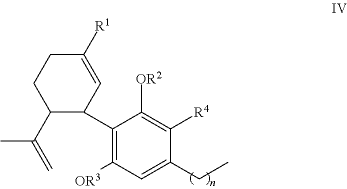

where R¹ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² and R³ are independently selected from hydrogen, methyl, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ acyl, linear or branched C₂-C₁₀ substituted acyl, an amine or amino acid, amino acid ester, R⁴ is selected from hydrogen, substituted or unsubstituted alkyl, carboxyl, alkoxy, aryl, aryloxy, arylalkyl, halo or amino, and n may an integer of 2 to 10 and the like. The compounds of Formula IV encompass stereoisomers, salts, and solvates thereof. In some embodiments, R² and R³ may, independently, be a linear or branched, substituted or unsubstituted C₂-C₁₀ acyl having a carboxylic acid terminus thereby producing a dicarboxylic acid, and salts thereof.

In some embodiments, R⁶ or R⁷ as illustrated in Formula I may be covalently connected to R³ to produce a cyclized moiety. For example, embodiments include compounds of general Formula V:

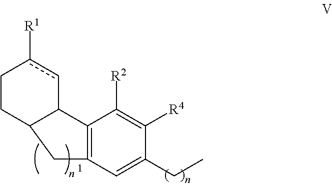

where R¹ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² is selected from hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, R⁴ is selected from hydrogen, substituted or unsubstituted alkyl, carboxyl, alkoxy, aryl, aryloxy, arylalkyl, halo or amino, and n and n¹ are each, individually, an integer of 2 to 10 and the like. The compounds of Formula V encompass stereoisomers, salts, and solvates thereof. In some embodiments, R² may be a linear or branched, substituted or unsubstituted C₂-C₁₀ acyl having a carboxylic acid terminus thereby producing a dicarboxylic acid, and salts thereof.

In some embodiments, R⁶ or R⁷ as illustrated in Formula I may be covalently connected to R³ to produce a cyclized moiety. For example, embodiments include compounds of general Formula VI:

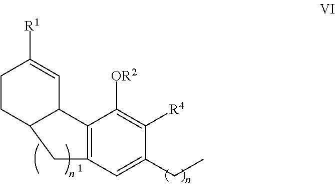

where R¹ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² is selected from hydrogen, methyl, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ acyl, linear or branched C₂-C₁₀ substituted acyl, an amine or amino acid, amino acid ester, R⁴ is selected from hydrogen, substituted or unsubstituted alkyl, carboxyl, alkoxy, aryl, aryloxy, arylalkyl, halo or amino. In various embodiments, n and n¹ are each, individually, an integer of 2 to 10 and the like. In some embodiments, the aliphatic chains created by n and n¹ may include one or more heteroatoms such as oxygen, nitrogen, sulfur. Additionally, a heteroatom can occupy the position at which the heterocycle is attached to the remainder of the molecule. The compounds of Formula VI encompass stereoisomers, salts, and solvates thereof. In some embodiments, R² may be a linear or branched, substituted or unsubstituted C₂-C₁₀ alkyl having a carboxylic acid terminus thereby producing a dicarboxylic acid, and salts thereof.

Unless otherwise stated, structures depicted above are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a carbon-13-enriched (¹³C-enriched) or carbon-14-enriched (¹⁴C-enriched) carbon are within the scope of this invention. Such compounds are useful, for example, as analytical tools, as probes in biological assays, or as therapeutic agents in accordance with the present invention.

In various embodiments, the compounds described above may be in a pharmaceutically acceptable salt form which, within the scope of sound medical judgment, is suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and —N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In various embodiments, the anti-inflammatory agents discussed above either by name or structure may be selected from any one or more of the anti-inflammatory agents discussed above either by name or structure.

The immunosuppressant, immunomodulator, anti-autoimmune, anti-allergy and/or anti-inflammatory agents disclosed herein may be provided in a composition (i.e., a pharmaceutical composition). Such agents may be present in the composition in a treatment effective amount as described elsewhere herein. In some embodiments, the composition may comprise the immunosuppressant, immunomodulator, anti-autoimmune, anti-allergy and/or anti-inflammatory agent in an amount of from about 50 w/w % to about 0.5 w/w %, relative to the total weight of the compositions, from about 30 w/w % to about 1 w/w %, relative to the total weight of the compositions, from about 20 w/w % to about 1 w/w %, relative to the total weight of the composition, from about 20 w/w % to about 5 w/w %, relative to the total weight of the composition, or any range or individual concentrations encompassed by these example ranges (e.g., about 1 w/w %, 5 w/w %, 10 w/w %, 15 w/w %, 20 w/w %, 30 w/w %, 40 w/w %, or 50 w/w %). In particular exemplary compositions, the immunosuppressant, immunomodulator, anti-autoimmune, anti-allergy and/or anti-inflammatory agent component may be present at from about 15 w/w % to about 10 w/w %, relative to the total weight of the composition, or any range or individual concentrations encompassed by this example range (e.g., about 10 w/w %, 11 w/w %, 12 w/w %, 13 w/w %, 14 w/w %, 15 w/w %).

In some embodiments, the compositions described herein may include a bioenhancer. As used herein, the term “bioenhancer” refers to an agent capable of enhancing bioavailability and efficacy of a drug with which it is co-administered, without any pharmacological activity of its own at the dose used. In some embodiments, the bioenhancers may also aid in the dissolution of the immunosuppressant, immunomodulator, anti-autoimmune, anti-allergy and/or anti-inflammatory agent. Bioenhancers include, for example, P-glycoprotein inhibitors, compounds that reverse P-glycoprotein-mediated efflux, limit metabolism of active agents, increase gastric emptying time and intestinal motility, reduce degradation of the active agent by hydrochloric acid, modify cell membrane permeability, produce a cholagogue effect, modify the bioenergetics and thermogenic properties of the active agent, suppress first pass metabolism, and inhibit metabolizing enzymes, stimulate gamma glutamyl transpeptidase, enhance the uptake of amino acids, and the like and combinations thereof. In some embodiments, the bioenhancers may be herbal or nutraceutical bioenhancers. Examples of bioenhancers encompassed by the invention include piperine, quercetin, genistein, naringin, sinomenine, glycyrrhizin, nitrile glycosides, cuminum, cyminum, zingiver, officinale, lysergol, Allium sativum, Aloe vera, and the like and combinations thereof.

In some embodiments, the bioenhancers may be liposomes, microspheres, nanoparticles, transfersomes, ethosomes, nanoemulsions, microemulsions, lipid based systems, polymeric micelle formulations, ketoprofen-loaded solid lipid nanoparticles, and the like, which may be made from beeswax, carnauba wax, or other natural waxes and solid lipids, and combinations thereof. In some embodiments, the bioenhancers may be liposomal enhancers such as, for example, Ginkgo biloba lipid-based systems, silybin lipid-based systems, ginseng lipid-based systems, hawthorn lipid-based systems, quercetin lipid-based systems, curcumin lipid-based systems, and the like and combinations thereof. In further embodiments, the bioenhancers may be capsaicin transfersomes, colchicine transfersomes, vincristine transfersomes, and the like and combinations thereof, which may find particular use as natural skin penetration agents.

In some embodiments, the bioenhancer may be a cyclic amine such as, for example, benzylamines, pyrrolidines, piperidines, morpholines, piperazines, and the like and derivatives thereof. In certain embodiments, the cyclic amine may be piperidine or a piperidine derivative such as N-methylpiperidine, N-ethylpiperidine, N-phenylpiperidine, pelletierine, sedamine, sedridine, allosedridine, dumetorine, solenopsins, isosolenopsins, andrachcinidine, cassine, prosopinine, prosophylline, morusimic acid, and the like and combinations thereof. In some embodiments, the cyclic amine may be isolated from or contained in an extract from a natural source such as pepper, sanguinarine, coptisine, goldenseal, and the like and combinations thereof. For example, piperine enhances bioavailability by modulating DNA receptor binding and cell signal transduction, while inhibiting efflux pumps that remove the active agent from cells. This inhibits drug metabolizing enzymes and stimulates absorption by stimulating gut amino acid transporters and inhibiting cellular pumps responsible for drug elimination from cells and intestinal production of glucuronic acid. Piperine also increases the absorption of the active agent in the gastrointestinal tract and inhibits enzymes responsible for drug metabolism especially in the liver during first pass metabolism such as hepatic aryl hydrocarbon hydrolase and UDP-glucuronyltransferase activities. Piperine modifies the rate of glucuronidation by lowering the endogenous UDP-glucuronic acid content and also by inhibiting transferase activity. Piperine inhibits P-glycoprotein and cytochrome P450 3A4, also CYP1A1, CYP1B1, CYP1B2, CYP2E1, CYP3A4, among others and makes target receptors more responsive to drugs, acting as receptors for drug molecules, increasing GIT vasculature by vasodilation to increase the absorption of drugs, modulation of cell membrane dynamics which increases transport of drugs across the cell membranes.

The amount of bioenhancer in the compositions of the invention may be from about 0.01 w/w % to about 20 w/w %, relative to the total amount of the composition or in some embodiments, from about 0.02 w/w % to about 10 w/w %, relative to the total weight of the composition, from about 0.02 w/w % to about 5 w/w %, relative to the total amount of the composition, from about 0.05 w/w % to about 2 w/w %, relative to the total amount of the composition, or any range or individual concentration encompassed by these example ranges.

Pharmaceutically acceptable carriers, adjuvants, or vehicles include not various compounds and compositions that allow for administration of the compounds without impacting the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally, by inhalation spray, topically, rectally, nasally, sublingually, buccally, vaginally or via an implanted reservoir. The term “parenteral” as used herein includes all routes of administration not involving the gastrointestinal tract, including sublingual, subcutaneous, intravenous, intramuscular, intra-articular, intra-synovial, intrasternal, intrathecal, intrahepatic, intralesional and intracranial injection or infusion techniques. In certain embodiments, the compositions may be administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution.

In some embodiments, fixed oils may be used as a solvent or suspending medium. Any bland fixed oil may be used for this purpose including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions may be administered in the form of suppositories for rectal administration. These may be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract may be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions may be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions may also be administered by nasal aerosol or inhalation. Such compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of the compound of various compounds described above may be combined with the carrier materials to produce a composition in a single dosage form will vary depending upon the host treated, the particular mode of administration. For example, in some embodiments, the compositions may be formulated to include the compounds in a dosage of between 0.01-100 mg/kg body weight, although a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. In some embodiments, the amount of an individual compound will depend on the pharmacological properties of the particular compound.

In some embodiments, the compositions described above may further include one or more pharmaceutically acceptable diluents, fillers, disintegrants, binders, lubricants, surfactants, emulsifiers, buffers, humectants, solubilizers, preservatives, colorants, plasticizers, and the like and combinations thereof. The person of ordinary skill in the art may refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of embodiments.

For example in some embodiments, the composition may include a buffering agent. Buffering agents may be used to provide drug stability, to control the therapeutic activity of the drug substance, and to prevent the initial discomfort associated with injections. Suitable buffers include, but are not limited to, sodium bicarbonate, sodium citrate, citric acid, sodium phosphate, and the like and combinations thereof. When one or more buffers are utilized in the formulations of the invention, they may be combined with a pharmaceutically acceptable vehicle and present in an amount from about 0.1 w/w % to about 20 w/w %.

In some embodiments, the composition may include an antioxidant. Such antioxidant may be, for example, butylated hydroxytoluene, ascorbic acid, ascorbyl palmitate, butylated hydroxyanisole, 2,4,5-trihydroxy butyrophenone, 4-hydroxymethyl-2,6-di-tert-butylphenol, crythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant may be present in a concentration of about 0.01 w/w % to about 1 w/w % of the total composition or any individual concentration encompassed by this example range.

Broadly speaking, the formulations may be prepared by combining together the components of the formulation, as described herein, at a temperature and for a time sufficient to provide a pharmaceutically acceptable composition. For example, in some embodiments, the components of the compositions may be dissolved, suspended, dispersed or otherwise mixed in a selected carrier or vehicle, at an effective concentration such that the condition to be treated is relieved or ameliorated.

The formulations of embodiments may include one or more immunosuppressant, immunomodulator, anti-autoimmune, anti-allergy and/or anti-inflammatory agents, such as those described above. In some embodiments, these agents may be the only active agents or primary active agents in the formulation. In other embodiments, the formulations may be a vaccine containing one or more immunosuppressant, immunomodulator, anti-autoimmune, anti-allergy and/or anti-inflammatory agents. A vaccine is a biological preparation that provides active acquired immunity to a particular infectious or malignant disease. In some embodiments, the vaccine may contain a component or impurity that resembles a disease-causing microorganism and may be made from weakened or killed forms of the microbe, its toxins, or one of its surface proteins. The agent stimulates the body's immune system to recognize the agent as a threat, destroy it, and to further recognize and destroy any of the microorganisms associated with that agent that it may encounter in the future. In some embodiments, the vaccine may be prophylactic (to prevent or ameliorate the effects of a future infection by a natural or “wild” pathogen), or therapeutic (to fight a disease that has already occurred, such as cancer).

In certain embodiments, the biologic may be a ribonucleic acid (RNA) vaccine that may direct the patient's cellular machinery to produce, for example, native proteins to antibodies or protein constructs that may have therapeutic activity inside and outside of cells. Such “mRNA vaccines” may be used to induce a balanced immune response against, for example, hMPV, PIV, RSV, MeV, BetaCoV, MERS-COV, SARS-COV, HCoV-OC43, HCoV-229E, HCoV-NL63, HCOV-NL, HCOV-NH, HCOV-HKUI, and the like and combinations thereof. In some embodiments, the mRNA vaccine may be directed to respiratory viruses such as hMPV RNA vaccines, PIV RNA vaccines, RSV RNA vaccines, MeV RNA vaccines, BetaCoV RNA vaccines, and any combination of two or more of hMPV RNA vaccines, PIV RNA vaccines, RSV RNA vaccines, MeV RNA vaccines, and BetaCoV RNA vaccines.

In some embodiments, the biologic may include at least one RNA polynucleotide having an open reading frame encoding at least one respiratory virus antigenic polypeptide, formulated in a cationic lipid nanoparticle. In some embodiments, the mRNA vaccine, may include at least one RNA polynucleotide having an open reading frame encoding at least one of hMPV, PIV, RSV, MeV, or a BetaCoV, MERS-COV, SARS-COV, HCoV-OC43, HCOV-229E, HCOV-NL63, HCOV-NL, HCOV-NH, HCOV-HKUI, antigenic polypeptides, and the like and combinations thereof. In some embodiments, ormRNA vaccines may include an RNA polynucleotide combined with the flagellin adjuvant, which may produce superior properties, for example, larger antibody titers. Thus, some embodiments include RNA (e.g., mRNA) vaccines that include at least one RNA (e.g., mRNA) polynucleotide having an open reading frame encoding at least one antigenic polypeptide or an immunogenic fragment thereof (e.g., an immunogenic fragment capable of inducing an immune response to the antigenic polypeptide) and at least one RNA (e.g., mRNA polynucleotide) having an open reading frame encoding a flagellin adjuvant.

In further embodiments, the biologic may be a nucleic acid (DNA or RNA) encoding at least one (e.g., at least 2, 3, 4 or 5) hMPV, PIV, RSV, MeV, and/or a BetaCoV, MERS-COV, SARS-COV, HCoV-OC43, HCOV-229E, HCOV-NL63, HCOV-NL, HCOV-NH, HCOV-HKUI, RNA polynucleotide.

In various embodiments, the antigenic polypeptide encoded by the RNA or DNA may be a major surface glycoprotein G or an immunogenic fragment thereof. In some embodiments, the antigenic polypeptide may be Fusion (F) glycoprotein (e.g., Fusion glycoprotein F0, F1 or F2) or an immunogenic fragment thereof. In some embodiments, the antigenic polypeptide may be a major surface glycoprotein G or an immunogenic fragment thereof and F glycoprotein or an immunogenic fragment thereof. In some embodiments, the antigenic polypeptide may be a nucleoprotein (N) or an immunogenic fragment thereof, phosphoprotein (P) or an immunogenic fragment thereof, large polymerase protein (L) or an immunogenic fragment thereof, matrix protein (M) or an immunogenic fragment thereof, small hydrophobic protein (SH) or an immunogenic fragment thereof nonstructural protein 1 (NS1) or an immunogenic fragment thereof, or nonstructural protein 2 (NS2) and an immunogenic fragment thereof.

Certain embodiments include administering agents for treating autoimmune and inflammatory diseases that affect molecular targets. For example, systemic steroids, although of demonstrated effectiveness in autoimmune and anti-inflammatory conditions, have been shown to weaken the immune response essential to effective immunization. Therefore, alternatives to the steroidal and other immunosuppressant drugs commonly administered in the treatment of chronic autoimmune conditions (e.g., lupus, RA, IBD, etc.) may be required to achieve beneficial outcomes in subjects reporting pathognomonic signs and symptoms following receipt of medicines containing nucleic acid (e.g., mRNA) components.

Several safe and effective agents that act upon pathways and targets have been identified that inhibit development of autoimmune and inflammatory responses within the host without eliminating the desired immunization effect. The agents of greatest therapeutic benefit were found to be those possessing individual and synergistic actions at novel autoimmune and/or anti-inflammatory targets, including, but not limited to, interleukins, such as, interleukin-1 (IL-1), interleukin-6 (IL-6), interleukin-17 (IL-17), and interleukin-23 (IL-23), TNF-α, Nrf2, INFγ, PPARγ, pregnane X receptor (PXR); CB1, CB2, and additional sites within the endocannabinoid system. Embodiments include administering any therapeutic agent that acts upon these molecular targets, producing an anti-autoimmune and/or anti-inflammatory response.

In some embodiments, the immunosuppressant, immunomodulator, anti-autoimmune, anti-allergy, or anti-inflammatory agent is a compound that acts via G protein-coupled receptors (GPCRs). GPCRs include, but are not limited to, cannabinoid receptor-1 (CB1), cannabinoid receptor-2 (CB2), and orphan GPCRs (e.g., GPR18, GPR35, GPR55, and GPR119). Other suitable immunosuppressant, immunomodulator, anti-autoimmune, anti-allergy, or anti-inflammatory agents may include compounds that act on targets within the endocannabinoid system, including, but not limited to activing via fatty acid amide hydrolases (FAAHs), transient receptor potential (TRP) channels, ligand and voltage-gated ion channels, and receptor heteromers including one or more of the aforementioned receptors.

The methods disclosed herein include administering an immunosuppressant, immunomodulator, anti-autoimmune, anti-allergy, or anti-inflammatory agent, or a composition containing an immunosuppressant, immunomodulator, anti-autoimmune, anti-allergy, or anti-inflammatory agent, to a patient in need of treatment. In some embodiments, the immunosuppressant, immunomodulator, anti-autoimmune, anti-allergy, or anti-inflammatory agent may be administered concurrently with the biologic. In some embodiments, concurrent administration may be carried out by administering a formulation containing both an immunosuppressant, immunomodulator, anti-autoimmune, anti-allergy, or anti-inflammatory agent and a biologic. In other embodiments, concurrent administration may be carried out by administering a biologic and an immunosuppressant, immunomodulator, anti-autoimmune, anti-allergy, or anti-inflammatory agent contemporaneously. Contemporaneous administration involves the separate administration of a first amount of am immunosuppressant, immunomodulator, anti-autoimmune, anti-allergy and/or anti-inflammatory agent and a second amount of a biologic sufficiently close in time to have the desired therapeutic effect, without limiting the order in which the agents are administered to the subject. In various embodiments, the immunosuppressant, immunomodulator, anti-autoimmune, anti-allergy, or anti-inflammatory agent may be one or more of the respective agents identified and described above. In other embodiments, the immunosuppressant, immunomodulator, anti-autoimmune, anti-allergy, or anti-inflammatory agent may be another immunosuppressant, immunomodulator, anti-autoimmune, anti-allergy, or anti-inflammatory agent known in the art.

In some embodiments, an immunosuppressant, immunomodulator, anti-autoimmune, anti-allergy, or anti-inflammatory agent may be administered before a biologic. For example, the immunosuppressant, immunomodulator, anti-autoimmune, anti-allergy, or anti-inflammatory agent may be administered 1 hour, 24 hours, 48 hours, 1 day, 2 days, 3, days, 5 days, 1 week, 2 weeks, 3 weeks, 1 month, 2 months, 6 months, or more before administration of the biologic. In other embodiments, the immunosuppressant, immunomodulator, anti-autoimmune, anti-allergy, or anti-inflammatory agent may be administered 1 hour, 24 hours, 48 hours, 1 day, 2 days, 3, days, 5 days, 1 week, 2 weeks, 3 weeks, 1 month, 2 months, 6 months, or more after administration of the biologic.

The patient may or may not exhibit symptoms of inflammatory, autoinflammatory or autoimmune disease (i.e. autoimmune-like symptoms). For example, in some embodiments, the patient may appear healthy when the immunosuppressant, immunomodulator, anti-autoimmune, anti-allergy, or anti-inflammatory agent is administered. For example, the patient in need of treatment may be an individual susceptible to a disease or malady, for example, an individual having a history or familial disposition to the disease or malady or that has been exposed to susceptibility factors, that may be treated using the compounds and compositions of the invention prior to the onset of symptoms. In other embodiments, administering an immunosuppressant, immunomodulator, anti-autoimmune, anti-allergy, or anti-inflammatory agent may occur after symptoms of inflammatory, autoinflammatory or autoimmune disease have developed. In some embodiments, administering may be carried out after symptoms have resolved, for example, to prevent or delay their recurrence.

Autoimmune-like or inflammatory symptoms include any symptoms known to be associated with autoimmune diseases and inflammatory disorders including, but are not limited to, joint pain and stiffness; muscle aches, pains, or weakness, fever; malaise; cutaneous manifestations (e.g., butterfly-shaped rash across the nose and checks; other skin rashes); unusual weight loss or weight gain; anemia; neurological or neuropsychiatric manifestations (e.g., trouble thinking, memory problems, confusion, depression, headache, seizures, strokes); kidney problems (e.g., nephritis, e.g., glomerulonephritis); chest pain; sun or light sensitivity; hair loss; Raynaud's phenomenon; vascular lesions or other vascular manifestations (e.g., Raynaud's phenomenon, nail fold telangiectasia and infarct, splinter hemorrhages, chilblain LE, acquired C1 esterase deficiency, vasculitis, urticarial vasculitis, purpura, thrombophlebitis, livedo reticularis, antiphospholipid syndrome, Degos syndrome and calcinosis)), as well as biological concomitants of lupus as disclosed herein or as known in the art (e.g., immune complexes, elevated levels of cytokines (e.g., interferons (e.g., Type I interferons, e.g., IFN-α and/or IFN-β); interleukins (e.g., IL-6, IL-8, IL-1, and IL-18) and TNF-α), elevated levels of antibodies associated with lupus (e.g., antinuclear antibodies (e.g., anti-Smith antibodies, anti-double stranded DNA (dsDNA) antibodies, anti-U1 RNP, SS-a (or anti-Ro), SS-b (or anti-La)), antiphospholipid antibodies, anti-ss DNA antibodies, anti-histone antibodies, or anticardiolipin antibodies), and overexpression of IFN inducible genes. Symptoms may be assessed using assays and scales (e.g., Systemic Lupus Activity Measure-Revised (SLAM-R) disclosed and the like and combinations thereof. In some embodiments, the inflammatory, autoinflammatory or autoimmune-like symptoms may include nephritis (e.g., glomerulonephritis, interstitial nephritis, perivascular inflammation, or protein cast severity), proteinuria, or spleen inflammation. In certain embodiments, the symptoms may include glomerulonephritis or an immune complex. In some embodiments, the inflammatory, autoinflammatory and/or autoimmune-like symptoms may include overexpression of pro-inflammatory cytokines (e.g., IFN-α, TNF-α, IL-6, IL-8, IL-1, IL-17, IL-23).

In some embodiments, the compounds (i.e., an immunosuppressant, immunomodulator, anti-autoimmune, anti-allergy, anti-inflammatory agent) and compositions of the invention may be administered using any amount and any route of administration effective for treating or lessening the severity of the inflammatory, autoinflammatory or autoimmune-like symptoms.

The compositions of the invention may be administered to humans and other animals using routes of administration including oral, rectal, parenteral, intracisternal, intravaginal, intraperitoneal, topical (as by powders, ointments, creams, liniments, or drops), sublingual, buccal, as an oral or nasal spray, and the like and combinations thereof depending on the severity of the infection being treated.

In certain embodiments, an effective amount of an immunosuppressant, immunomodulator, anti-autoimmune, anti-allergy and/or anti-inflammatory agent for administration one or more times a day to a 70 kg adult human may comprise about 0.0001 mg to about 4000 mg, about 0.0001 mg to about 3000 mg, about 0.0001 mg to about 200 mg, about 0.001 mg to about 1500 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of the agent per unit dosage form. In certain embodiments, formulations of the agent may be at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

In some embodiments, a single dose may be sufficient to treat or prevent inflammatory, autoinflammatory or autoimmune symptoms associated with administration of a biologic (e.g., vaccine), which may be delivered in one or more aliquots to achieve the desired dose. In other embodiments, multiple doses may be required to treat or prevent inflammatory, autoinflammatory or autoimmune symptoms associated with administration of a biologic. Dosing may occur at intervals of minutes, hours, days, weeks, months or years or continuously over any one of these periods. The administered amount may be an amount sufficient to treat or alleviate the inflammatory, autoinflammatory or autoimmune symptoms associated with administration of the biologic.

The exact amount of the compounds required will vary from subject to subject, and may depend, for example, on the species, age, and general condition of the subject, the severity of the disease, the particular agent, its mode of administration, and the like. The compounds of the invention are preferably formulated in dosage unit form for case of administration and titration to effect and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, genetic profile, sex, gender identity, gender preference, and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions may also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid are used in the preparation of injectables.

The injectable formulations may be sterilized, for example, by filtration through a bacterial-retaining filter, or by incorporating sterilizing agents in the form of sterile solid compositions which may be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsulated matrices of the compound in biodegradable polymers such as polylactide-polyglycolide. Depending upon the ratio of compound to polymer and the nature of the particular polymer employed, the rate of compound release may be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which may be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, caplets, troches, lozenges, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, c) solution retarding agents such as paraffin, f) absorption accelerators such as quaternary ammonium compounds, g) wetting agents such as, for example, cetyl alcohol and glycerol monostearate, h) adsorbents and/or absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may be used as fillers in soft and hard-filled gelatin capsules using such excipients as lactose or milk sugar as well as high molecular weight polyethylene glycols and the like. The solid dosage forms of tablets, dragees, capsules, pills, and granules may be prepared with coatings and shells such as enteric coatings and other coatings well known in the pharmaceutical formulating art. They may contain opacifying agents and may also be of a composition that releases the active ingredient(s) only in a certain part of the intestinal tract, optionally, in a delayed manner. Examples of embedding compositions that may be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds may also be in micro-encapsulated form with one or more excipients as noted above. The solid dosage forms of tablets, dragées, capsules, pills, and granules may be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and may also be of a composition that releases the active ingredient(s) only, or preferentially, in a certain part of the intestinal tract, optionally, in a delayed manner. Examples of embedding compositions that may be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms may be made by dissolving or dispensing the compound in the proper medium. Absorption enhancers may also be used to increase the flux of the compound across the skin. The rate may be controlled by either providing a rate controlling membrane or by dispersing the compound in a polymer matrix or gel.

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated”.

In some embodiments, the compositions may be delivered topically by, for example, applying standard or bioenhanced compositions directly to the skin of a patient in need for treatment at the location of the injury or disease. In other embodiments, the compositions may be applied locally to the skin of a patient before, for example, surgery, injections, or other medically necessary injury. In further embodiments, the compositions may be administered transdermally, percutaneously, or by microneedle injection. Administration may also be, for example, intravenous, intraperitoneal, subdermal, subcutaneous, intradermal, transcutaneous, intramuscular, oral, intra-joint, parenteral, intranasal, or by inhalation. Suitable sites of administration thus include, but are not limited to, the skin, bronchium, gastrointestinal tract, eye, buccal cavity, and ear.

In embodiments such as those described above, the compounds and compositions of the invention may be administered one or more times each day, and administering may be carried out for a period of at least 1 month, 2 months, 3 months, 4 months, 6 months, 8 months, 12 months, or indefinitely depending on the condition of the patient or disease or injury being treated. In some embodiments, the treatment comprises a 1 week to a 6 month course of treatment, or a 1 week to a 5 year course of treatment. In some embodiments, the composition may be administered once, as needed, once daily, twice daily, three times a day, once a week, twice a week, every other week, every other day, or the like. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In some embodiments, the immunosuppressant, immunomodulator, anti-autoimmune, anti-allergy and/or anti-inflammatory agent, and the biologic (e.g., vaccine) may be contained in a kit. The kit may include, for example, the immunosuppressant, immunomodulator, anti-autoimmune, anti-allergy and/or anti-inflammatory agent, and the biologic, each packaged or formulated individually, or packaged or formulated in combination. Thus, the immunosuppressant, immunomodulator, anti-autoimmune, anti-allergy and/or anti-inflammatory agent may be present in first container, and the biologic may be present in a second container. The container or containers may be placed within a package, and the package may optionally include administration or dosage instructions. The kits disclosed herein may comprise the immunosuppressant, immunomodulator, anti-autoimmune, anti-allergy and/or anti-inflammatory agent, and the biologic, in a form or forms suitable for administration (e.g., in one or more pharmaceutical compositions comprising pharmaceutically acceptable carriers, diluents, adjuvants and/or excipients). The kits may optionally comprise instructions describing a method of using the immunosuppressant, immunomodulator, anti-autoimmune, anti-allergy and/or anti-inflammatory agent in one or more of the methods described herein (e.g., for treating or preventing an inflammatory, autoinflammatory and/or autoimmune symptom associated with administration of the biologic).

Those skilled in the art will be aware that the invention described herein is subject to variations and modifications other than those specifically described. It is to be understood that the invention described herein includes all such variations and modifications. The invention also includes all such steps, features, methods, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1

A formulation for treating inflammation or autoimmune-like conditions associated with the administration of a biologic may include one or more of the components in Table 1.

TABLE 1

| | | |
|---|---|---|
| 30 | ml | hemp seed oil |
| 1.8 | g | cannabidiol (CBD, cannabinoid) |
| 40 | mg | quercetin (bioenhancer) |
| 200 | ml | liquid diluent |

Hemp seed oil in the formulation of Table 1 is used as carrier oil and may include any number of fatty acid components. The components of the hemp seed oil used in this Example 1 are presented in Table 2.

TABLE 2

| | |
|---|---|
| 16% | omega-3-fatty acid |
| 15% | alpha-linoleic acid |
| 1% | stearidonic acid |
| 53% | omega-6-fatty acid |
| 4% | gamma-linolenic acid |
| 11% | omega-9-fatty acid |

The composition of Table 1 may be orally administered to a patient after administration of a biologic to relieve or ameliorate inflammation and autoimmune-like symptoms associated with the biologic. Administration may be repeated on a daily basis until the symptoms have been reduced or eliminated.

### Example 2

A formulation for treating inflammation or autoimmune-like conditions associated with the administration of a biologic may include the components in Table 3.

TABLE 3

| | |
|---|---|
| 135-400 mg/day | Magnesium (as the ascorbate salt) |
| 200-500 mg, 1-3 times/day | Quercetin |
| 250-1,000 mg, 1-2 times/day | Vitamin C (as an ascorbate salt) |
| 750-3,200 IU/day | Vitamin D3 |
| 250-1,000 mg/day | Omega-3 Fatty Acids |
| 300-600 mg, 1-3 times/day | Fulvic Acid |

The composition of Table 3 may be orally administered to a patient after administration of a biologic to relieve or ameliorate inflammation and autoimmune-like symptoms associated with the biologic. Administration may be repeated on a daily basis until the symptoms have been reduced or eliminated.

### Example 3

A formulation for treating inflammation or autoimmune-like conditions associated with the administration of a biologic may include one or more of the components in Table 4.

TABLE 4

| | | |
|---|---|---|
| 1.5-10 | mg/kg/day | Garcinoic Acid |
| 0.2 | mg/kg/day | Glycyrrhizin |
| 50-250 | mg/day | Bacillopeptidase F |
| 2.5-15 | mg/kg/day | Cannabidiol |
| 1-3 | g/day | Curcumin |
| 13-132 | mg/day | β-Caryophyllene |

The composition of Table 4 may be orally administered to a patient after administration of a biologic to relieve or ameliorate inflammation and autoimmune-like symptoms associated with the biologic. Administration may be repeated on a daily basis until the symptoms have been reduced or eliminated.

### Example 4

A formulation for treating inflammation or autoimmune-like conditions associated with the administration of a biologic may include one or more of the components in Table 5.

TABLE 5

| | |
|---|---|
| 325-500 mg every 4-6 hours | Acetaminophen |
| 25-75 mg every 4-6 hours | Diphenhydramine |
| 200-400 mg/day | Hydroxychloroquine |
| 160-3,200 mg/day in divided doses | Lactoferrin |
| 500-1,000 mg, 1-2 times/day | Resveratrol |

The composition of Table 5 may be orally administered to a patient after administration of a biologic to relieve or ameliorate inflammation and autoimmune-like symptoms associated with the biologic. Administration may be repeated on a daily basis until the symptoms have been reduced or eliminated.

### Example 5

Following administration of a biologic (e.g., an mRNA vaccine), a patient may exhibit one or more of the signs and symptoms of an inflammatory or autoimmune response in Table 6 within 6 hours to several weeks after administration.

TABLE 6
Swelling of glands and/or dependent extremities
Constitutional symptoms, e.g., fatigue, lethargy, anergia, malaise,
myasthenia
Mood alterations of unknown etiology, unrelated to triggering events or
medications
Joint pain and/or swelling, rigidity or reduced mobility
Recurring fever of unknown origin or etiology (non-infectious)
Abdominal pain or gastrointestinal complaints of unknown etiology
Respiratory complaints, including coughing fits, dyspnea (shortness of
breath) with activity or at rest, etc.
Skin problems related to inflammation, irritation, rash, itching,
desquamation
Cardiac symptoms, including pounding, or fluttering rapid heartbeats,
exertional chest pain, or after heavy meals.

In a method of preventing an inflammatory, autoinflammatory and/or autoimmune symptom associated with administration of the biologic, the patient may be treated concurrently, as a pre-medication, and/or as a follow-up adjuvant therapy to the biologic, one or more the immunosuppressant, immunomodulator, anti-autoimmune, anti-allergy and/or anti-inflammatory agents of Tables 3-5 at the specified dosage, with optional supplementation with one or more of the additional agents of Tables 3-5 as indicated. The patient is expected to exhibit a gradual resolution of autoimmune and/or inflammatory symptoms commencing within hours to days.

In a method of treating an inflammatory, autoinflammatory and/or autoimmune symptom associated with administration of the biologic, a patient who complains of symptoms consistent with, or related to, the list of autoimmune and inflammatory signs and symptoms in Table 6 above may be subsequently administered a single or extended course of treatment with one or more the immunosuppressant, immunomodulator, anti-autoimmune, anti-allergy and/or anti-inflammatory agents of Tables 3-5 at the specified doses, with optional supplementation with one or more of the additional agents in Tables 3-5 as indicated. The patient is expected to exhibit a gradual resolution of autoimmune and/or inflammatory symptoms commencing within hours or days.

### Example 6

A middle-aged, previously healthy patient complaining of acute inflammatory symptoms including abnormal fatigue, joint stiffness, and abdominal rash within several days after receiving a COVID-19 vaccine or mRNA biologic therapy can be administered a two-week course of 400 mg/day hydroxychloroquine, optionally supplemented with 1000 mg/day omega-3 fatty acids, for several days to several weeks until reported symptoms remit. The patient is expected to obtain partial or full relief from these symptoms within several days of commencing this course of treatment. Such treatment may be extended safely until the patient's desired therapeutic outcome is achieved.

1. A method of treating or preventing an inflammatory, autoinflammatory and/or autoimmune symptom associated with administration of a biologic to a subject, the method comprising administering to the subject an effective amount of a composition containing an immunosuppressant, immunomodulator, anti-autoimmune, anti-allergy, anti-inflammatory agent, or a combination thereof.

2. The method of claim 1, wherein the immunosuppressant, immunomodulator, anti-autoimmune, anti-allergy, or anti-inflammatory agent is selected from the group consisting of tamibarotene, N-acetylcysteine, CDP7657, hydroxychloroquine, B cell depletion, anti-CD20 therapy, rituximab, proteasome inhibitors, carfilzomib, bortezomib, immunoproteasome inhibitors, ONX 0914, toll-like receptor inhibitors, hydroxychloroquine, IMO-3100, DV1179), nonsteroidal anti-inflammatory drugs (NSAID), corticosteroids, and combinations thereof.

3. The method of claim 1, wherein the immunosuppressant, immunomodulator, anti-autoimmune, anti-allergy, or anti-inflammatory agent is selected from the group consisting of laquinimod, rapamycin, cyclophosphamide (Cytoxan), azathioprine (Imuran, Azasan), mycophenolate (Cellcept), leflunomide (Arava), methotrexate (Trexall), CNTO 136, and combinations thereof.

4. The method of claim 1, wherein the immunosuppressant, immunomodulator, anti-autoimmune, anti-allergy, or anti-inflammatory agent is selected from the group consisting of AGS-009, rontalizumab (rhuMAb IFNalpha), vitamin D3, sifalimumab (MEDI-545), AMG 811, IFNα kinoid, CEP33457, and combinations thereof.

5. The method of claim 1, wherein the immunosuppressant, immunomodulator, anti-autoimmune, anti-allergy, or anti-inflammatory agent is selected from the group consisting of anti-B-cell agents, Epratuzumab, LY2127399, Ocrelizumab, Atacicept, A-623, SBI-087 and an anti-T-cell agent (such as AMG557).

6. The method of claim 1, wherein the immunosuppressant, immunomodulator, anti-autoimmune, anti-allergy, or anti-inflammatory agent is a compound that acts via G protein-coupled receptors (GPCRs), fatty acid amide hydrolases (FAAHs), transient receptor potential (TRP) channel inhibitors, ligand and voltage-gated ion channels, and combinations thereof.

7. The method of claim 1, wherein the biologic is an mRNA-based vaccine.

8. The method of claim 7, wherein the mRNA-based vaccine is a COVID-19 vaccine.

9. The method of claim 1, wherein administration of the composition to the subject is carried out concurrently with administration of the biologic to the subject.

10. The method of claim 1, wherein administration of the composition to the subject is carried out from about 1 day to about 6 months after administration of the biologic to the subject.

11. The method of claim 1, wherein administration of the composition to the subject comprises administration of a single dose of the composition to the subject.

12. The method of claim 1, wherein administration of the composition to the subject comprises a 1 week to a 6 month course of treatment.

13. The method of claim 1, wherein administration of the composition to the subject comprises a 1 week to a 5 year course of treatment.

14. A method of treating or preventing an inflammatory, autoinflammatory and/or autoimmune symptom associated with administration of COVID-19 vaccine to a subject, the method comprising administering to the subject an effective amount of a composition containing an immunosuppressant, immunomodulator, anti-autoimmune, anti-allergy, anti-inflammatory agent, or combinations thereof.

15. The method of claim 14, wherein the subject exhibits an inflammatory, autoinflammatory and/or autoimmune symptom selected from the group consisting of joint pain, joint stiffness, muscle aches, pains, or weakness, fever; malaise, skin rashes, weight loss or weight gain, anemia, trouble thinking, memory problems, confusion, depression, headache, seizures, strokes, kidney problems, nephritis, glomerulonephritis, chest pain, sun or light sensitivity, hair loss, Raynaud's phenomenon, vascular lesions, nail fold telangiectasia and infarct, splinter hemorrhages, chilblain LE, acquired C₁ esterase deficiency, vasculitis, urticarial vasculitis, purpura, thrombophlebitis, livedo reticularis, antiphospholipid syndrome, Degos syndrome and calcinosis, Guillain-Barré syndrome, Functional Neurological Disorder (FND), elevated levels of cytokines, elevated levels of antibodies associated with lupus, overexpression of interferon-inducible genes, and combinations thereof.

16. The method of claim 14, wherein the immunosuppressant, immunomodulator, anti-autoimmune, anti-allergy, or anti-inflammatory agent is selected from the group consisting of tamibarotene, N-acetylcysteine, CDP7657, hydroxychloroquine, B cell depletion, anti-CD20 therapy, rituximab, proteasome inhibitors, carfilzomib, bortezomib, immunoproteasome inhibitors, ONX 0914, toll-like receptor inhibitors, hydroxychloroquine, IMO-3100, DV1179), nonsteroidal anti-inflammatory drugs (NSAID), corticosteroids, and combinations thereof.

17. The method of claim 14, wherein the immunosuppressant, immunomodulator, anti-autoimmune, anti-allergy, or anti-inflammatory agent is selected from the group consisting of laquinimod, rapamycin, cyclophosphamide (Cytoxan), azathioprine (Imuran, Azasan), mycophenolate (Cellcept), leflunomide (Arava), methotrexate (Trexall), CNTO 136, and combinations thereof.

18. The method of claim 14, wherein the immunosuppressant, immunomodulator, anti-autoimmune, anti-allergy, or anti-inflammatory agent is selected from the group consisting of AGS-009, rontalizumab (rhuMAb IFNalpha), vitamin D3, sifalimumab (MEDI-545), AMG 811, IFNα kinoid, CEP33457, and combinations thereof.

19. The method of claim 14, wherein the immunosuppressant, immunomodulator, anti-autoimmune, anti-allergy, or anti-inflammatory agent is selected from the group consisting of anti-B-cell agents, Epratuzumab, LY2127399, Ocrelizumab, Atacicept, A-623, SBI-087 and an anti-T-cell agent (such as AMG557).

20. The method of claim 14, wherein the immunosuppressant, immunomodulator, anti-autoimmune, anti-allergy, or anti-inflammatory agent is a compound that acts via G protein-coupled receptors (GPCRs), fatty acid amide hydrolases (FAAHs), transient receptor potential (TRP) channel inhibitors, ligand and voltage-gated ion channels, and combinations thereof.

21. The method of claim 14, wherein administration of the composition to the subject is carried out concurrently with administration of the biologic to the subject.

22. The method of claim 14, wherein administration of the composition to the subject is carried out from about 1 day to about 6 months after administration of the biologic to the subject.

23. The method of claim 14, wherein administration of the composition to the subject comprises administration of a single dose of the composition to the subject.

24. The method of claim 14, wherein administration of the composition to the subject comprises a 1 week to a 6 month course of treatment.

25. The method of claim 14, wherein administration of the composition to the subject comprises a 1 week to a 5 year course of treatment.

26. Use of an immunosuppressant, immunomodulator, anti-autoimmune, anti-allergy, anti-inflammatory agent, or a combination thereof, in the manufacture of a medicament for treating or preventing an inflammatory, autoinflammatory and/or autoimmune symptom associated with administration of a biologic to a subject.

27. Use of an immunosuppressant, immunomodulator, anti-autoimmune, anti-allergy, anti-inflammatory agent, or a combination thereof, in the manufacture of a medicament for treating or preventing an inflammatory, autoinflammatory and/or autoimmune symptom associated with administration of a COVID-19 vaccine to a subject.
